(19) United States
(12) Patent Application Publication
(10) Pub. No.: US 2005/0245848 A1
(43) Pub. Date: Nov. 3, 2005

(54) Muscle strength measuring method and device

(76) Inventor: Yves Chatrenet, Sallanches (FR)

Correspondence Address: WILLIAM H. EILBERG, THREE BALA PLAZA, SUITE 501 WEST, BALA CYNWYD, PA, 19004, US

(21) Appl. No.: 10/526,098

(22) PCT Filed: Aug. 29, 2003
(86) PCT No.: PCT/FR03/02604

§ 371 Date: Feb. 25, 2005

(30) Foreign Application Priority Data
Sep. 2, 2002 (FR) 0211082

(51) Int. Cl. A61B 5/103; A61B 5/117

(52) U.S. Cl. 600/587; 600/592; 600/595

(57) ABSTRACT

The invention relates to a muscle strength measuring method and device. The inventive device includes a lower support base (1) on which a patient (2) stands with his/her weight bearing on the plantar surface of the foot/feet. An upper vertically-movable support bracket (5) rests vertically on the head (7) of the patient (2). A position sensor (12) is used to measure the vertical position of the upper support bracket (5) while a lift force sensor (13) is used to measure the lift force applied by the head (7) of the patient (2) against the upper support bracket (5). Plantar support sensors (4a, 4b) are used to monitor the plantar force exerted by the patient (2) on the lower support base (1). The patient tends to grow in height through the use of the short, deep back muscles and the device can be used to measure the effects of self stretching in terms of ranges of values and forces.

### TECHNICAL FIELD OF THE INVENTION

The present invention relates to apparatus and methods for testing the muscular capacity of the muscles of the back, in particular of the short deep muscles.

Backache is frequently associated with a lack of muscular capacity of the muscles for balancing the vertebral column.

Two types of muscles are involved in this extensor muscles, which are relatively long superficial muscles, used for movements of large amplitude, and short deep muscles, in particular for stretching the vertebral column.

The extensor muscles have already been studied and devices have been designed for measuring and testing them.

The short deep muscles have been neglected, however, and the testing apparatus and methods known in the art are unable to test them.

### STATEMENT OF THE INVENTION

The present invention aims essentially to test the muscular capacity of the short deep back muscles by causing them to work in self-stretching mode.

The documents FR 2 661 600 and EP 1 183 996 describe devices for measuring the length of the human body. Such devices are intended to measure the size of a patient, either standing or seated. These devices cannot automatically test the position of the patient while measuring his size or measure a self-stretching force.

The short deep back muscles stretch the vertebral column and measuring the stretch depends greatly on the position of the feet of the patient: a standing patient may effect a false stretching by modifying his normal plantar position, for example by standing on tip-toe (plantar flexion), by raising the front portion of the foot (dorsal flexion), by flexing the toes downwards, by rolling the foot outwards (supination). The invention aims to avoid or at least to detect any such false stretching, so as to measure the real stretching reliably and reproducibly.

Thus the principal problem addressed by the invention is that of designing means for evaluating reliably and simply the muscular capacity of the short deep back muscles.

A subsidiary problem addressed by the invention is that of evaluating the muscular capacity of the psoas major muscle under conditions ensuring that this test is reliable. The problem is then to place the patient in a reproducible position allowing repetitive and reliable evaluation of the muscular capacity of the psoas major muscle.

Reliable testing necessitates in particular that the body segments are always in a defined position when testing the capacity of a muscle acting on those segments.

The invention exploits the particular position that the patient must adopt when testing the short deep back muscles also and reliably to test the psoas major muscle, which is the muscle used to raise the thigh forwards.

To achieve the above and other objects, the invention proposes a device for measuring muscle strength, comprising :
a lower support base adapted to support a standing patient in plantar support on said lower support base,
an upper support bracket movable vertically above the lower support base and conformed to bear vertically on the head of said patient,
means for selectively immobilizing the upper bearing bracket in vertical position,
means for measuring the vertical position of the upper support bracket,
means for measuring the vertical lifting force that the head of the patient applies to the upper support bracket,
plantar support sensors in the lower support base adapted to test for maintained normal plantar support of the foot or feet of the patient by producing a signal if the foot or feet are no longer in normal plantar support.

With normal plantar support, a patient tends to increase his height by working the short deep back muscles. His head bears on the bottom of the upper support bracket, which is either immobilized vertically to measure the self-stretching force or allowed to slide vertically to measure the amplitude of the self-stretching. The device can therefore be used to measure the effects of self-stretching in terms of amplitude and in terms of force.

In practice, the upper support bracket is carried by a vertical column connecting it to the lower support base.

Alternatively, the upper support bracket may be carried by a vertical wall near which the lower support base stands.

In one particular embodiment, the lower support base comprises plantar support sensors adapted to ensure that the bearing force is greater than a predefined minimum in both the anterior area and the posterior area of the foot.

In one particular embodiment allowing supplementary testing of the psoas major muscle, the device further comprises an anterior support which constitutes a frontal bearing against which the anterior base of the thigh of the patient can bear on flexing by less than 30°, preferably less than 20°, with means for measuring the forward muscular force to evaluate the frontal bearing force of the thigh of the patient.

A preferred embodiment of the device comprises a computation unit associated with memory means and display means and receiving signals from the vertical position measuring means and the plantar support sensors of the lower bearing base, the memory means containing a stored program for controlling the computation unit, the stored program including in particular a self-stretching amplitude measuring sequence for storing vertical positions of the upper support bracket when the latter is allowed to slide freely and is pushed by the head of the patient and a self-stretching muscular force measuring sequence for storing values of the lifting force applied by the head of the patient to the upper support bracket when said upper support bracket is immobilized in an appropriate vertical position.

The program may advantageously include an endurance measurement sequence for measuring the time for which an appropriate lifting force applied by the head of the patient to the upper support bracket is maintained.

Another aspect of the invention consists in a method of measuring the muscular strength of a patient using the above device and comprising the steps of :

a) placing the patient in a standing position on the lower support base,

b) ensuring continuously that the patient is in normal plantar support on the lower bearing base, and interrupting the measurement if the plantar support is not normal,

c) measuring the amplitude of self-stretching of the patient by allowing the upper support bracket to slide up and down according to vertical movements of the head of the patient and storing successive positions of the upper support bracket,

d) determining the maximum self-stretching value corresponding to the highest position recorded during the previous step,

e) fixing the vertical position of the upper support bracket a few millimeters below the maximum self-stretching value,

f) measuring the self-stretching forces by storing the lifting force exerted by the head of the patient on the upper support bracket when the latter is immobilized vertically.

An advantageous embodiment of the method further comprises the steps of :

g) recording the maximum lifting force,

h) selecting a lifting force threshold lower than the maximum lifting force,

i) determining the maximum endurance time (TME) by measuring the maximum time for which a lifting force greater than or equal to the lifting force threshold is maintained.

The method may instead or additionally further comprise the steps of :

g) storing the maximum lifting force,

h) selecting a lifting force threshold below the maximum lifting force,

j) in an intermittent contraction (TCI) mode, generating an intermittent signal detectable by the patient to prompt alternate self-stretching contractions and relaxations and counting the number of contractions reaching a lifting force threshold after a sufficient relaxation characterized by a sufficiently low lifting force.

Finally, for testing the psoas major muscle, the method may advantageously further comprise the steps of :

k) with the patient in the position of the preceding steps for measuring the self-stretching force, in normal plantar support on one foot and the other leg bent, measuring the maximum frontal force with which the thigh of the bent leg of the patient bears against an anterior support,

l) selecting a frontal bearing force threshold less than the maximum frontal bearing force exerted by the thigh,

m) determining the endurance of the psoas major muscle by measuring the maximum time for which a frontal bearing force greater than or equal to the frontal bearing force threshold is maintained and/or by counting the number of alternating contractions in periods of relaxation reaching the frontal bearing force threshold.

### BRIEF DESCRIPTION OF THE DRAWINGS

Other objects, features and advantages of the present invention will emerge from the following description of particular embodiments of the invention, which is given with reference to the appended drawings, in which :

FIG. 1 is a diagrammatic side view showing a patient in a testing position on one embodiment of a device of the present invention for measuring muscular strength ;

FIG. 2 is a plan view of the lower support base, showing the preferred areas for placing bearing sensors for testing plantar support ;

FIG. 3 is a diagrammatic side view showing a patient in a position for testing the psoas major muscle on a second embodiment of a device of the present invention ;

FIG. 4 is a diagram of the essential measurement and test units and their connections to a central computation unit ;

FIG. 5 shows one particular embodiment of means for testing normal plantar support of a patient and

FIG. 6 shows another embodiment of means for testing normal plantar support of a patient.

### DESCRIPTION OF THE PREFERRED EMBODIMENTS

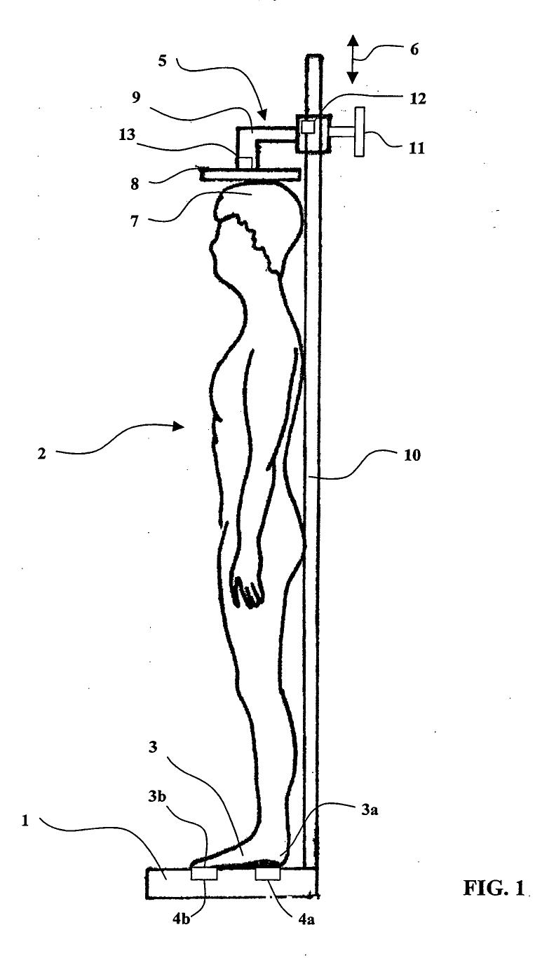

In the embodiment shown in FIG. 1, a device of the invention for measuring muscular strength comprises a lower support base 1 adapted to support a patient 2 who stands on said lower support base 1 by virtue of plantar support for his feet 3.

The lower support base 1 is essentially a horizontal plate provided with means for testing continuing normal plantar support of the foot or feet 3 of the patient 2. The object is to be sure that the foot 3 is resting on the lower support base 1 through the sole of the foot, with no stretching effect resulting, for example, from raising of the heel 3a by plantar flexion, raising of the toes 3b of the foot by dorsal flexion, flexing of the ankles and/or supination of the foot.

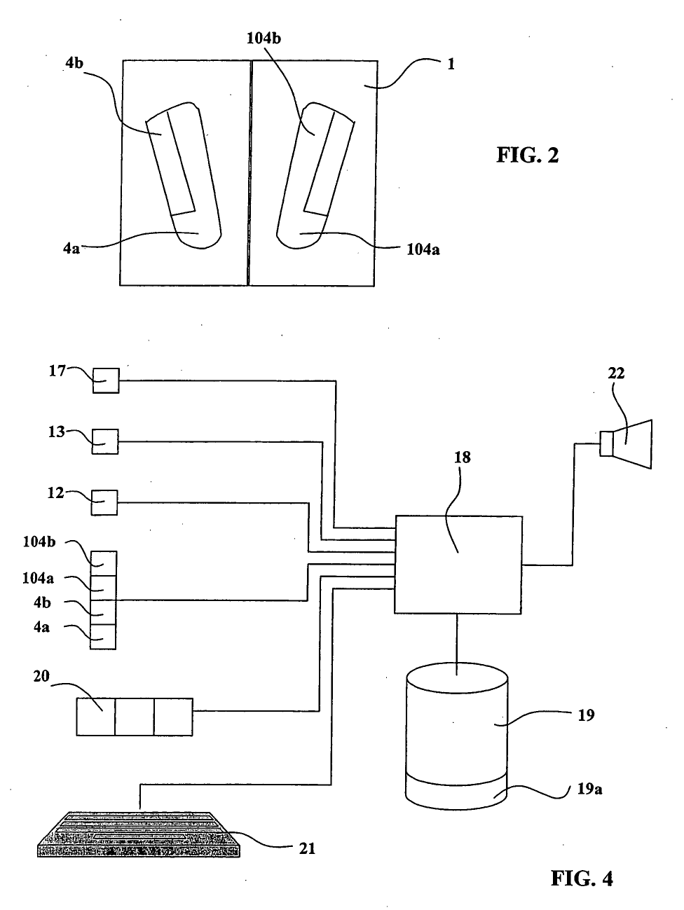

In a simplified embodiment, bearing sensors 4a may be placed under the area of the heel 3a and bearing sensors 4b may be placed under the toes 3b, for example, with means for ensuring that the force measured by the sensors 4a and 4b does not fall below a predefined minimum threshold, for example a threshold equal to a few Newtons. FIG. 2 shows one possible distribution of the plantar support sensors, with the above sensors 4a and 4b for a first foot of the patient and plantar support sensors 104a and 104b for the patient's other foot. Other embodiments are described hereinafter.

The device further comprises an upper support bracket 5 movable vertically above the lower support base 1, as indicated by the arrow 6.

The upper support bracket 5 is conformed to rest vertically on the head 7 of the patient 2 and to this end comprises a horizontal plate 8 held by an arm 9, for example.

In the embodiment shown in FIG. 1, the upper support bracket 5 is mounted so that the posterior end of the arm 9 slides along a vertical column 10 that connects it to the lower support base 1. A locking screw 11 is used to selectively immobilize the upper support bracket 5 in a vertical position selected by the operator.

The upper support bracket 5 is associated with a position sensor 12 for measuring the vertical position of the upper support bracket 5.

One or more lifting force sensors 13 placed in an appropriate manner on the upper support bracket 5 measure a vertical lifting force applied by the head 7 of the patient 2 to the upper support bracket 5.

Alternatively, the lifting force sensors 13 may be placed at the interface between the column 10 and the upper support bracket 5, or in an intermediate region of the connection between the lower support base 1 and the upper support bracket 5, or even between the floor and the lower support base 1. In this latter case, the lifting force is determined as the difference between the weight measured in the absence of any lifting force and the weight measured in the presence of a lifting force.

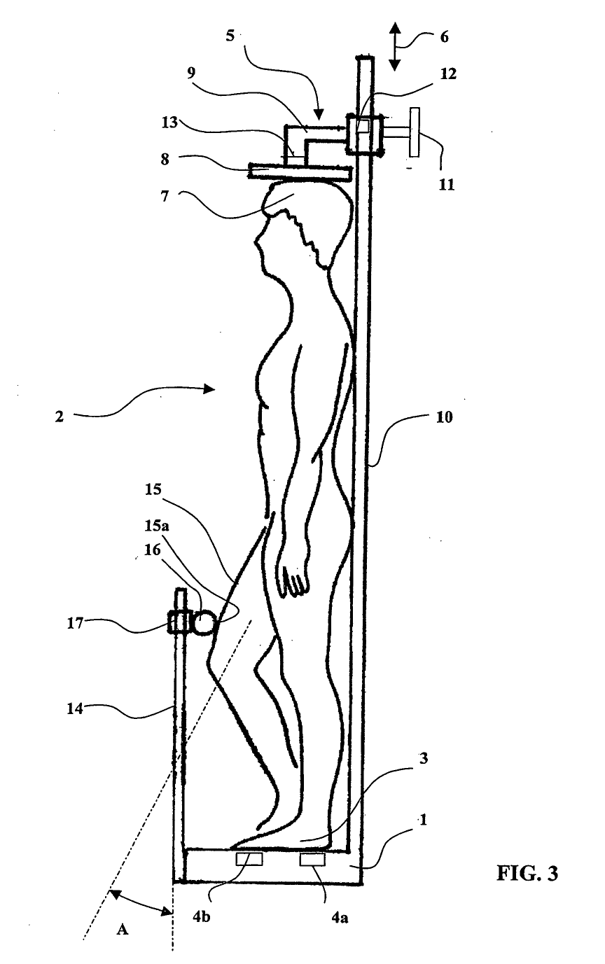

The FIG. 3 embodiment comprises the same components as the FIG. 1 embodiment, which are identified by the same reference numbers. There is therefore no need to describe them again.

In the FIG. 3 embodiment, the device further comprises an anterior support 14 adapted to constitute a front support against which the anterior base 15a of the thigh 15 of the patient 2 may bear in a position in which the thigh 15 is bent at an angle A of less than 30° and preferably less than 20°.

The anterior support 14 comprises a transverse bearing bar 16 and a front force sensor 17 to evaluate the forward force with which the thigh 15 of the patient 2 bears on it.

The height of the transverse bearing bar 16 is preferably adjustable by sliding it along the anterior support 14 and immobilizing it to adapt it to the morphology of the patient 2.

Refer now to FIG. 4, which shows the plantar support sensors 4a and 4b, the position sensor 12, the lifting force sensor 13, the front force sensor 17 and the similar plantar support sensors 104a and 104b for the patient's other foot.

All these sensors produce output signals that are sent over corresponding lines to a computation unit 18 such as a microcontroller or a microprocessor. The computation unit 18 is associated with memory means 19, output display means 20, and preferably data entry means such as a keyboard 21. The computation unit 18 is preferably also connected to a signal generator 22 producing signals perceptible by the patient 2, for example a sound or light signal generator. The memory 19 includes a program area 19a for storing a program.

The program stored in the program area 19a contains in particular a self-stretching measurement sequence that stores successive vertical positions of the upper support bracket 5 when it slides freely when pushed by the head 7 of the patient 2 and a sequence for measuring maximum self-stretching muscle forces by storing values of the lifting force applied by the head 7 of the patient 2 to the upper support bracket 5 when said upper support bracket 5 is immobilized by the screw 11 in an appropriate vertical position.

The program may advantageously comprise an endurance measurement sequence for measuring the time for which an appropriate lifting force is applied by the head 7 of the patient 2 to the upper support bracket 5.

During the self-stretching measurement sequence, the computation unit 18 examines the signals produced by the position sensor 12, stores the successive vertical positions of the upper support bracket 5 in the memory 19 and determines the maximum value of the lifting force or maximum self-stretching value.

The operator then immobilizes the upper support bracket 5 in a vertical position a few millimeters below the maximum self-stretching value by tightening the screw 11. During the sequence for measuring the self-stretching muscle forces which follows on from the previous self-stretching measurement sequence, the computation unit 18 examines the signals coming from the lifting force sensor 13 and stores in the memory 19 the sequence of values of the lifting force applied by the head 7 of the patient 2 to the upper support bracket 5. The program then determines the maximum value of the lifting force.

A lifting force threshold below the maximum lifting force is then chosen, either automatically by the program stored in the program area 19a or manually by intervention of the operator at the keyboard 21.

During the next endurance measurement sequence, the computation unit 18 examines the signals coming from the lifting force sensor 13 and measures the maximum time for which a lifting force greater than or equal to the lifting force threshold previously defined is maintained.

This determines a maximum endurance time (TME). The endurance measurement ceases when the lifting force is below the lifting force threshold for more than three seconds.

The device may also be used in an intermittent contraction (TCI) mode. In this case, the stored program contains sequences of instructions used by the computation unit 18 to control the signal generator 22 so that it generates an intermittent signal detectable by the patient 2 to prompt alternate self-stretching contractions and relaxations. The computation unit examines the signals coming from the lifting force sensor 13 and counts the number of contractions reaching the lifting force threshold after a sufficient relaxation characterized by the lifting force sensor 13 detecting a sufficiently low lifting force.

In the case of a device for measuring the muscular strength of the psoas major muscle, as shown in FIG. 3, the computation unit 18 examines the signals coming from the front force sensor 17 and measures the forces with which the thigh 15 of the patient 2 bears against the anterior support 14. The computation unit 18 then determines the maximum forward bearing force stored in the memory 19, after which a frontal bearing force threshold below the stored maximum frontal bearing force is chosen.

The program stored in the program area 19a includes a psoas major muscle endurance determination sequence during which the computation unit 18 examines the signals coming from the front force sensor 17, measures the maximum time for which a frontal bearing force greater than or equal to the frontal bearing force threshold is maintained, and/or counts the number of contractions reaching the frontal bearing force threshold and alternating with periods of relaxation.

For use of the device defined hereinabove, the invention provides a method of measuring the muscular force of a patient comprising a series of steps involving:

a) placing the patient 2 in a standing position with his two feet on the lower support base 1,

b) ensuring continuously that the patient 2 is in normal plantar support on the lower bearing base 1, that is to say bearing sufficiently both on his toes 3b and on his heels 3a, and interrupting the measurement if the plantar support is not normal,

c) placing the upper support bracket on the head 7 of the patient 2, and measuring the amplitude of self-stretching of the patient 2 by allowing the upper support bracket 5 to slide up and down according to vertical movements of the head 7 of the patient 2 and storing successive positions of the upper support bracket 5,

d) determining the maximum self-stretching value corresponding to the highest position recorded during the previous step,

e) fixing the vertical position of the upper support bracket 5 a few millimeters, for example 5 mm, below the maximum self-stretching value,

f) measuring the self-stretching forces by storing the lifting force exerted by the head 7 of the patient 2 on the upper support bracket 5.

To determine the maximum endurance time (TME), the following steps then follow:

g) recording the maximum lifting force,

h) selecting a lifting force threshold lower than the maximum lifting force,

i) determining the maximum endurance time by measuring the maximum time for which a lifting force greater than or equal to the lifting force threshold is maintained.

For the intermittent contraction (TCI) mode, the method comprises the following steps

g) storing the maximum lifting force,

h) selecting a lifting force threshold below the maximum lifting force,

j) in an intermittent contraction (TCI) mode, generating an intermittent signal detectable by the patient to prompt alternate self-stretching contractions and relaxations and counting the number of contractions reaching a lifting force threshold after a sufficient relaxation characterized by a sufficiently low lifting force.

To test the psoas major muscle, the method of the invention further comprises the following steps:

k) with the patient in the position for measuring the self-stretching force as described above, but in normal plantar support on only one foot, the other leg being bent so that the front base 15a of the thigh bears on a transverse support bar 16, measuring the maximum frontal force with which the thigh 15 of the patient 2 bears against the anterior support 14,

l) selecting a frontal bearing force threshold less than the maximum frontal bearing force exerted by the thigh 15,

m) determining the endurance of the psoas major muscle by measuring the maximum time for which a frontal bearing force greater than or equal to the frontal bearing force threshold is maintained and/or by counting the number of alternating contractions in periods of relaxation reaching the frontal bearing force threshold.

When testing short deep muscles by self-stretching, the efficacy of the invention is the result of permanently testing the position of the patient 2, and in particular his normal plantar support on both feet.

The simplified embodiment described with reference to FIGS. 1 and 3 is able to detect certain false stretching movements.

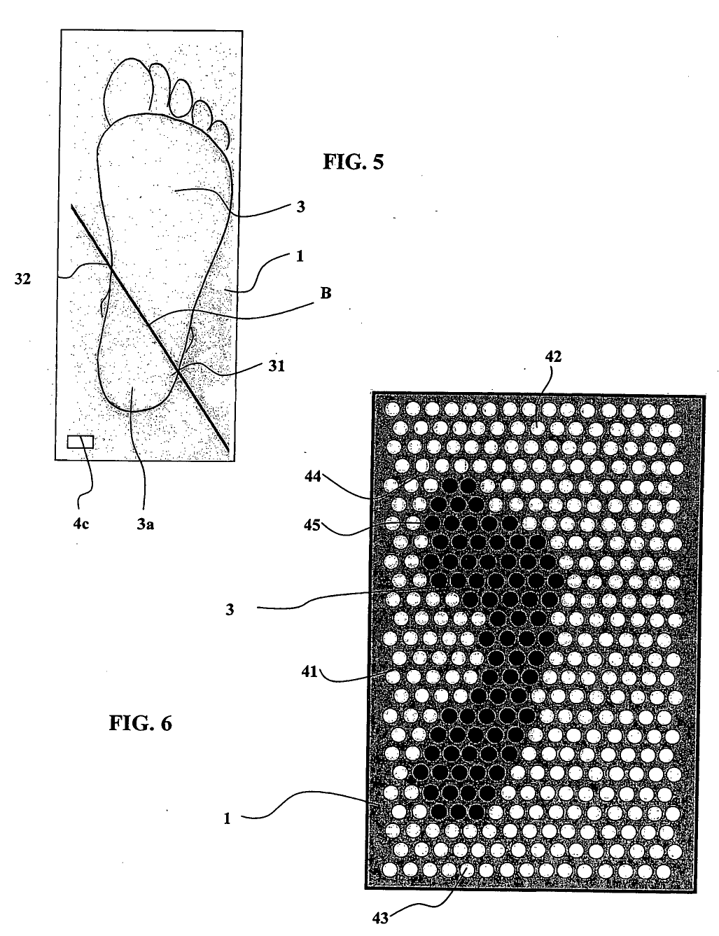

Improved means described hereinafter with reference to FIGS. 5 and 6 may be used to detect a greater number of false self-stretching movements.

As shown in FIG. 5, each foot rests on a lower support base 1 below which is an integral oblique tilt shaft B. The tilt shaft B is oriented obliquely at an angle of approximately 30° to 60°, so that the foot 3 is positioned above the shaft B, with the shaft B crossing the external posterior edge 31 of the heel 3a and an intermediate region 32 of the inside edge of the foot 3. A contact or proximity sensor 4c is provided under the posterior inside portion of the lower support base 1. Any attempt at plantar flexing and/or supination of the foot leads to tilting of the lower support base 1 and a loss of contact detected by the contact or proximity sensor 4c, which inhibits the self-stretching measurement.

In an improved embodiment, shown in FIG. 6, each support platform, such as the lower support base 1, comprises a matrix of sensors or contacts such as the sensors or contacts 41, 42, 43, 44 or 45 distributed over the surface of the lower support base 1 on which the foot 3 rests. Each sensor or contact 41-45 may have an area of approximately 1 cm² and be of a mechanical, electrical, thermal, photosensitive, capacitive or other type. Each sensor 41-45 is connected to the computation unit 18 (FIG. 4) and produces a pressure measurement signal that is analyzed by the computation unit. The program stored in the memory 19a controls the computation unit 18 so that it periodically scans each of the sensors 41-45. At the start of the measurement, the computation unit 18 is therefore able to determine the area on which the foot 3 is resting, or area shown in black in FIG. 6, corresponding to all of the sensors receiving pressure from the foot. Any subsequent loss of contact or pressure at one or more sensors initially activated by the normal plantar support inhibits operation of the system for measuring self-stretching forces.

The main sensors are positioned under the lower and inside portion of the foot, on the one hand, and under the heel, on the other hand.

One option is for the program to determine the resultant of all the pressures on the sensors for each foot, and varying beyond a tolerance defined by the operator of the system. Any modification of the plantar support varies this resultant, and variation beyond a tolerance defined by the operator of the system inhibits the system for measuring self-stretching forces.

A sensor 44 placed under the second phalange of the great toe and a sensor 45 placed under the head of the first metatarsal respectively detect extension of the great toe, upward movement of the head of the first metatarsal upon flexing the toes downwards, and also detect dorsal flexing of the foot. Deactivation of one or the other of the two sensors 44 and 45 inhibits the system for measuring self-stretching forces.

Clearly the device shown in FIG. 6 can detect all false stretching movements.

Intermediate devices for detecting only certain false stretching movements may naturally be envisaged. For example, a contact or sensor that is moved by sliding it along the inside edge of each platform may be positioned above the first toe. Stretching of the great toe activates this contact and inhibits the system for measuring self-stretching forces.

When testing the psoas major muscle, the accuracy of the invention is also the result of the permanent testing of self-stretching of the patient 2, in particular the position of his pelvis, from which his stretching more particularly results.

The present invention is not limited to the embodiments that have been described explicitly but includes diverse variants and generalizations thereof within the scope of the following claims.

1-11. (canceled)

12. A device for measuring muscle strength, comprising:
a lower support base adapted to support a standing patient in plantar support on said lower support base,
an upper support bracket movable vertically above the lower support base and conformed to bear vertically on the head of said patient,
means for selectively immobilizing the upper bearing bracket in vertical position,
means for measuring the vertical position of the upper support bracket,
means for measuring the vertical lifting force that the head of the patient applies to the upper support bracket, and
plantar support sensors in the lower support base adapted to test for maintained normal plantar support of the foot or feet of the patient by producing a signal if the foot or feet are no longer in normal plantar support.

13. The device according to claim 12, having a vertical column which carries the upper support bracket and connects it to the lower support base.

14. The device according to claim 12, wherein the lower support base comprises plantar support sensors adapted to ensure that the bearing force is greater than a predefined minimum in both the anterior area and the posterior area of the foot.

15. The device according to claim 12, further comprising an anterior support adapted to constitute a frontal bearing against which the anterior base of the thigh of the patient can bear on flexing by less than 30°, preferably less than 20°, with means for measuring the forward muscular force to evaluate the frontal bearing force of the thigh of the patient.

16. The device according to claim 12, comprising a computation unit associated with memory means and display means and receiving signals from the vertical position measuring means and the plantar support sensors of the lower bearing base, the memory means containing a stored program for controlling the computation unit, the stored program including in particular a self-stretching amplitude measuring sequence for storing vertical positions of the upper support bracket when the latter is allowed to slide freely and is pushed by the head of the patient and a self-stretching muscular force measuring sequence for storing values of the lifting force applied by the head of the patient to the upper support bracket when said upper support bracket is immobilized in an appropriate vertical position.

17. The device according to claim 16, wherein the stored program includes an endurance measurement sequence for measuring the time for which an appropriate lifting force applied by the head of the patient to the upper support bracket is maintained.

18. The device according to claim 16, wherein the stored program includes sequences of instructions for testing in an intermittent contraction mode by generating an intermittent signal detectable by the patient to prompt alternate self-stretching contractions and relaxations and counting the number of contractions reaching a lifting force threshold after a sufficient relaxation characterized by a sufficiently low lifting force.

19. A method of measuring the muscular strength of a patient using a device according to claim 12, comprising the steps of:
a) placing the patient in a standing position on the lower support base,
b) ensuring continuously that the patient is in normal plantar support on the lower bearing base, and interrupting the measurement if the plantar support is not normal,
c) measuring the amplitude of self-stretching of the patient by allowing the upper support bracket to slide up and down according to vertical movements of the head of the patient and storing successive positions of the upper support bracket,
d) determining the maximum self-stretching value corresponding to the highest position recorded during the previous step,
e) fixing the vertical position of the upper support bracket a few millimeters below the maximum self-stretching value,
f) measuring the self-stretching forces by storing the lifting force exerted by the head of the patient on the upper support bracket when the latter is immobilized vertically.

20. The method according to claim 19, further comprising the steps of:
g) recording the maximum lifting force,
h) selecting a lifting force threshold lower than the maximum lifting force,
i) determining the maximum endurance time by measuring the maximum time for which a lifting force greater than or equal to the lifting force threshold is maintained.

21. The method according to claim 19, further comprising the steps of:
g) storing the maximum lifting force,
h) selecting a lifting force threshold below the maximum lifting force,
j) in an intermittent contraction mode, generating an intermittent signal detectable by the patient to prompt alternate self-stretching contractions and relaxations and counting the number of contractions reaching a lifting force threshold after a sufficient relaxation characterized by a sufficiently low lifting force.

22. The method according to claim 19, further comprising the steps of:
k) with the patient in the position of the preceding steps for measuring the self-stretching force, in normal plantar support on one foot, measuring the maximum frontal force with which the thigh of the patient bears against the anterior support,
l) selecting a frontal bearing force threshold less than the maximum frontal bearing force exerted by the thigh,
m) determining the endurance of the psoas major muscle by measuring the maximum time for which a frontal bearing force greater than or equal to the frontal bearing force threshold is maintained and/or by counting the number of alternating contractions in periods of relaxation and reaching the frontal bearing force threshold.
